(19) United States
(12) Patent Application Publication
(10) Pub. No.: US 2019/0321290 A1
(43) Pub. Date: Oct. 24, 2019

(54) COMPOSITION AND METHOD FOR INHALATION

(71) Applicant: MannKind Corporation, Westlake Village, CA (US)

(72) Inventors: Joseph J. Guarneri, Stamford, CT (US); Nikhil Amin, Chappaqua, NY (US); Marshall L. Grant, Newtown, CT (US); John J. Freeman, New Fairfield, CT (US); Kelly S. Kraft, Poughquag, NY (US)

(21) Appl. No.: 16/434,938

(22) Filed: Jun. 7, 2019

Related U.S. Application Data
(63) Continuation-in-part of application No. 15/418,388, filed on Jan. 27, 2017.
(60) Provisional application No. 62/289,095, filed on Jan. 29, 2016.
(60) Provisional application No. 62/682,109, filed on Jun. 7, 2018.

(51) Int. Cl. A61K 9/00; A61M 15/00; A61M 16/14; A61K 31/216; A61K 9/16

(52) U.S. Cl. CPC A61K 9/0075; A61M 15/003; A61M 16/14; A61M 2202/064; A61K 9/1617; A61K 9/1623; A61K 31/216

(57) ABSTRACT

A prostaglandin composition and method for treating pulmonary arterial hypertension is disclose. The composition is based on diketopiperazine for pulmonary inhalation.

### CROSS REFERENCE TO RELATED APPLICATIONS

This application is a continuation-in-part of U.S. patent application Ser. No. 15/418,388, filed on Jan. 27, 2017, which claims the benefit of U.S. Provisional Patent Application Ser. No. 62/289,095, filed on Jan. 29, 2016, the entire contents each of which are incorporated herein by reference.

This application claims the benefit of U.S. Provisional Patent Application Ser. No. 62/682,109, filed on Jun. 7, 2018, the entire contents of which are incorporated herein by reference.

### TECHNICAL FIELD

Compositions and methods for treating pulmonary arterial hypertension are disclosed.

### BACKGROUND

Pulmonary arterial hypertension (PAH) is a complex, multifactorial, progressive syndrome characterized by persistent elevation of pulmonary artery pressure and pulmonary vascular resistance (PVR) that leads to increase in right ventricular afterload and eventually culminates in right heart failure. Right ventricular failure limits cardiac output during exertion. The most common symptom at presentation is breathlessness, fatigue, angina, syncope, and abdominal distension, with impaired exercise capacity as a hallmark of the disease.

The symptoms of PAH are non-specific. The symptoms at rest are reported only in very advanced cases due to the non-specific nature of the symptoms, there is a substantial delay of more than 2 years in the diagnosis of pulmonary hypertension (PH). Unfortunately approximately 70% of the patients with PH are diagnosed when they have reached an advanced stage of disease (World. Health Organization (WHO) Functional Class III and IV). Early identification and treatment of pulmonary hypertension (PH) is generally suggested because advanced disease may be less responsive to therapy. Treatment begins with a baseline assessment of disease severity, followed by primary therapy.

Assessing patients with pulmonary hypertension involves evaluating the severity of their disease using a range of clinical assessments, exercise tests, detection of specific biochemical markers, and echocardiographic and hemodynamic assessments. The clinical assessment of the patient has a pivotal role in the choice of the initial treatment, the evaluation of the response to therapy, and the possible escalation of therapy if needed.

PAH is classified into five groups (1-5) depending on the severity of the disease. In group 1, for example, the disease is heritable and commonly induced by drugs and toxins. PAH includes idiopathic pulmonary arterial hypertension (IPAH, formerly called primary pulmonary hypertension), hereditary PAH, or PAH due to diseases such as connective tissue diseases, HIV infection, portal hypertension, congenital heart disease, schistosomiasis, and drug or toxin exposure (e.g. anorexigens). Estimated prevalence PAH is 15-50 cases per million, in the USA and Europe. However, the prevalence of PAH in certain at-risk groups is substantially higher. For example, in HIV-infected patients the prevalence of PAH is 0.5%, in patients with collagen vascular disorders it has been reported to be 7-120%, and in patients with sickle cell disease the prevalence is around 2-3.75%. In patients with hepatosplenic schistosomiasis 5% may have PAH. It is estimated that 10% of adults with congenital heart disease (CHD) may also have PAH. PAH in the newborn, known as persistent pulmonary hypertension of the newborn has been estimated to occur in 0.2% of live-born term infants.

Group 2 patients develop PH due to left heart disease from, inter glia, left ventricular systolic dysfunction, left ventricular diastolic dysfunction, valvular disease, or congenital/acquired left heart inflow/outflow tract obstruction, and congenital cardiomyopathies. In group 3, the PH is due to chronic lung disease and/or hypoxia exhibiting chronic obstructive pulmonary disease, interstitial lung disease, other pulmonary diseases with mixed restrictive and obstructive pattern, sleep-disordered breathing, alveolar hypoventilation disorders, chronic exposure to high altitude and developmental lung diseases. In group 4, PH is due to chronic thromboembolic pulmonary hypertension and group 5 patients exhibit PH due to unclear multifactorial mechanisms, including hematologic disorders such as chronic hemolytic anemia, myeloproliferative disorders, splenectomy; systemic disorders such as sarcoidosis, pulmonary histiocytosis, lymphangioleiomyomatosis; metabolic disorders, including glycogen storage disease, Gaucher's disease and thyroid disorders; and other disorders such as tumor/mass obstruction, fibrosing mediastinitis, chronic renal failure, segmental PH.

Primary therapy is directed at the underlying cause of the PH and is warranted in nearly all patients with PH. The disease severity should be reassessed following primary therapy, in order to determine whether advanced therapy is indicated. Advanced therapy is directed at the pulmonary hypertension itself, rather than the underlying cause of the PH. Advanced therapy is widely accepted for many patients with group 1 pulmonary arterial hypertension (PAH). In contrast, it should only be administered on a case-by-case basis for patients with group 3 PH, group 4 PH, or group 5 PH, after carefully weighing the risks versus the benefits. Advanced therapy should not be administered to most patients with group 2 PH.

Until 2001, the only drug available to treat PAH was epoprostenol (Flolan, GlaxoSmithKline Pharmaceuticals), and it was mostly used as a bridge to transplantation. Since then, other therapies have evolved, and as a result the prognosis of patients with PAH has significantly improved.

The clinical assessment of the patient has a pivotal role in the choice of the initial treatment, the evaluation of the response to therapy, and the possible escalation of therapy if needed. As mentioned above, diagnosing patients with pulmonary hypertension involves evaluating the severity of their disease using a range of clinical assessments, exercise tests, identification of biochemical markers, echocardiographic and hemodynamic assessments.

The clinical severity of PAH is classified according to a system originally developed for heart failure by the New York Heart Association (NYHA) and then modified by WHO for patients with PH. This functional classification (I-IV) system links symptoms with activity limitations, and allows clinicians to quickly predict disease progression and prognosis, as well as the need for specific treatment regimens, irrespective of the underlying etiology of PAH. Class I patients exhibit PH, but without resulting limitation of physical activity, and ordinary physical activity does not cause dyspnea or fatigue, chest pain, or near syncope. Class lI patients exhibit pulmonary hypertension resulting in slight limitation of physic& activity. They are comfortable at rest and ordinary physical activity causes undue dyspnea or fatigue, chest pain, or near syncope. Class III are patients with pulmonary hypertension resulting in marked limitation of physical activity, they are comfortable at rest and less than ordinary activity causes undue dyspnea or fatigue, chest pain, or near syncope. Class IV are patients with pulmonary hypertension with inability to carry out any physical activity without symptoms. These patients manifest signs of right heart failure. Dyspnea and/or fatigue may even be present at rest, and discomfort is increased by any physical activity.

The pathogenesis of PH is complex and many biochemical pathways and cell types have been identified or proposed as contributing to this vasoconstriction and vascular remodeling. These include altered synthesis of nitric oxide (NO), prostacyclin (PGI) and endothelin (ET-1), impaired potassium channel and growth factor receptor function, altered serotonin transporter regulation, increased oxidant stress, and enhanced matrix production of vasoactive factors, calcium signaling molecules, inflammatory mediators, growth factors, bone morphogenetic protein receptor (BMPR2) mutations. However, the relative importance of each of these processes is unknown.

Clinical and preclinical studies strongly suggest that the pulmonary vascular endothelium plays a critical role and interactions between pulmonary endothelial cells with pulmonary arterial smooth muscle cells, and pulmonary pericytes plays a critical role in either initiation and/or perpetuation of the characteristic progressive pulmonary arterial obstruction in PAH. Pulmonary vascular endothelium is a critical local source of several key mediators for vascular remodeling, including growth factors (fibroblast growth factor [FGF]-2, serotonin [5-HT], angiotensin II, and vasoactive peptides (NO, PGI2, ET-1), cytokines (IL-1, IL-6, macrophage migration inhibitory factor [MIF]), and chemokines (monocyte chemoattractant protein [MCP]-1), adipokines (leptin). Endothelial dysfunction, is believed to occur early in disease and this leads to chronically impaired production of vasodilator and antiproliferative agents such as NO and prostacyclin, along with overexpression of vasoconstrictor and proliferative substances such as thromboxane A2 and endothelin-1. Paracrine overproduction of ET-1, 5-HT, angiotensin II, and FGF-2 contributes to an increased pulmonary vascular cell proliferation, survival, migration, and differentiation. Many of these abnormalities both elevate vascular tone and promote endothelial and smooth muscle cell proliferation followed by structural changes or remodeling of the pulmonary vascular bed, which in turn results in an increase in pulmonary vascular resistance. in addition, in the adventitia there is increased production of extracellular matrix including collagen, elastin, fibronectin, and tenascin.

Over the past two decades, three main mechanistic pathways, namely the endothelin, nitric oxide and prostacyclin (prostaglandin (PG) 12) pathways are targeted for PAH-specific therapies. The PAH-specific drug classes include the endothelin receptor antagonists, phosphodiesterase type-5 inhibitors (PDE-5i), including bosentan, sitaxsentan and ambrisentan and others such as sildenafil, tadalafil, or soluble guanylate cyclase stimulators and prostanoids. These “targeted” therapies have led to both short- and long-term benefits to many patients. All of the currently approved PAH drugs belong to one of these classes. These agents have received their initial regulatory approval as monotherapy for the primary indication by improving six-minute walk distance (6MWD). Additional endpoints such as functional class, hemodynamics, and clinical worsening of PAH have also been included in most of these Phase III trials. In these registration trials, drugs in these classes have been universally shown to improve exercise capacity and haemodynamics of patients with PAH. In addition, some of these drugs were shown to be associated with improvements in outcome for patients with PAH compared with historical data.

All of the currently approved PAH drugs belong to one of these classes, including, protenoids, for example, epoprostenol (Flolan® and Veletri® intravenous infusions), treprostinil (Remodulin® subcutaneous/IV infusion); Tyvaso® (inhaled ×4 time.day), Iloprost ® (inhaled 6-9 times/day). These agents have received their initial regulatory approval as monotherapy for the primary indication by improving six-minute walk distance (6MWD). Additional endpoints such as functional class, hemodynamics, and clinical worsening of PAH have also been included in most of these Phase III trials. In these registration trials, drugs in these classes have been universally shown to improve exercise capacity and haemodynamics of patients with PAH. In addition, some of these drugs were shown to be associated with improvements in outcome for patients with PAH compared with historical data.

Parenteral prostacyclin analogs have been the most widely studied. Intravenous epoprostenol was the first US Food and Drug Administration (FDA)-approved treatment for PAH (approved in 1995). However, due to its extremely short half-life (3-5 min), epoprostenol needs to be delivered as a continuous intravenous infusion through an indwelling catheter, with the risk of rebound PAH and acute right heart failure in case of infusion interruption. Furthermore, due to the inherent chemical instability of epoprostenol at room temperature and neutral pH (room temperature stability <8 hours), ice packs are needed to slow decomposition throughout the infusion period. A thermostable epoprostenol preparation for infusion (Veletri®), which does not require cooling, has been approved for use by the FDA. However, serious adverse events related to the delivery system include pump malfunction, local site infection, catheter obstruction, and sepsis continues to be a barrier for its use.

Treprostinil, is a longer-acting tricyclic benzidine analogue of epoprostenol with a terminal elimination half-life of approximately 2 to 4 hours and a distribution half-life of approximately 40 minutes. Unlike epoprostenol, Treprostinil is chemically stable at room temperature allowing it to be administered at ambient temperature and overcomes some of the limitations associated with epoprostenol therapy. Treprostinil causes vasodilation of pulmonary and systemic arterial vascular beds, and inhibits platelet aggregation by binding to prostacyclin IP receptors located on the surface of vascular smooth muscle cells and platelets. Treprostinil (Remodulin®) was first approved by the FDA in 2002 for adults with WHO group 1 PAH and functional class II to class IV status for continuous subcutaneous infusion and is marketed by United Therapeutics (Silver Spring, Md.). In a pivotal 12 week randomized, controlled trial of 470 patients, subcutaneous Treprostinil significantly improved exercise capacity compared with placebo. The most common adverse events noted in subcutaneous infusion of Treprostinil-treated patients were infusion site pain.

Currently, an oral, extended release tablet of treprostinil diolamine (Orenitran®) is also available. However, with orally delivered medications, the absorption of treprostinil may be inconsistent particularly taken with food. The pharmacological and physiochemical properties of treprostinil make this drug amenable to intermittent administration via the inhaled route. Tyvaso® and Iloprost (Ventavis®) are solutions for inhalation, which need to be administered using a special nebulizer for a prolonged period of time and often times in a physician's office. Using Tyvaso® inhalation system [Opti-Neb ultrasonic nebulizer (NebuTec, Elsenfeld, Germany)]. The inhalation system is complex to assemble and use, cumbersome to administer the dose (patient need to reset the device 3 times during a treatment session after every 3 breaths) and was found to have high error rates in human factor study. There is a distinct risk of under dosing as patient need to take 9 breaths within a specified 90 second time limit. Additionally, breath counter mechanism is triggered by time (time related) and not by inspiration or expiration flow or effort (breath related) and thus patient can overdose or under dose themselves by taking more or less than prescribed breaths (dose) in the 90 seconds time limit. The system also requires 4 different cleaning schedule (daily, weekly, monthly and yearly). Accordingly, new methods of PAH treatment are needed to facilitate the administration of these products to a patient.

Drug delivery to lung tissue has been achieved using a variety of devices for inhalation, including, nebulizers and inhalers, such as metered dose inhalers and dry powder inhalers to treat local disease or disorders. Dry powder inhalers used to deliver medicaments to the lungs contain a dose system of a powder formulation usually either in bulk supply or quantified into individual doses stored in unit dose compartments such as hard gelatin capsules or blister packs. Bulk containers are equipped with a measuring system operated by the patient in order to isolate a single dose from the powder immediately before inhalation.

Dosing reproducibility with inhalers requires that the drug formulation is uniform and that the dose be delivered to a subject with consistency and reproducible results. Therefore, the dosing system ideally should operate to completely discharge all of the formulation effectively during an inspiratory maneuver when the patient is taking his/her dose. However, complete powder discharge from the inhaler is not required as long as reproducible dosing can be achieved. Flow properties of the powder formulation, and long term physical and mechanical stability in this respect, are more critical for bulk containers than they are for single unit dose compartments. Good moisture protection for preventing product degradation can be achieved more easily for unit dose compartments such as blisters. However, the materials used to manufacture the blisters allow air into the drug compartment and subsequently, the formulation loses viability with prolonged storage, particularly if the formulation to be delivered is hygroscopic. The ambient air permeating through the blisters carries in humidity that destabilizes the active ingredient. Additionally, dry powder inhalers which use blisters to deliver a medicament by inhalation can suffer with inconsistency of dose delivery to the lungs due to variations in geometry of the air conduit architecture resulting from puncturing films or peeling films of the blisters.

Dry powder inhalers such as those described in U.S. Pat. Nos. 7,305,986, 7,464,706, 8,499,757 and 8,636,001, which disclosures are incorporated herein by reference in their entirety, can generate primary drug particles, or suitable inhalation plumes during an inspiratory maneuver by deagglomerating the powder formulation within a capsule or cartridge comprising a single dose. The amount of fine powder discharged from the inhaler's mouthpiece during inhalation is largely dependent on, for example, the inter-particulate forces in the powder formulation and the efficiency of the inhaler to separate those particles so that they are suitable for inhalation. The benefits of delivering drugs via the pulmonary circulation are numerous and include rapid entry into the arterial circulation, avoidance of drug degradation by liver metabolism, and ease of use without discomfort.

Some dry powder inhaler products developed for pulmonary delivery have met with some success to date. However, due to lack of practicality and/or cost of manufacture, there is room for improvement. Some of the persistent problems observed with prior art inhalers, include lack of device ruggedness, inconsistency in dosing, inconvenience of the equipment, poor deagglomeration, problems with delivery in light of divorce from propellant use, high manufacturing costs, and/or lack of patient compliance. Therefore, the inventors have identified the need to design and manufacture new formulations and inhalers with consistent improved powder delivery properties, easy to use, and having discrete configurations which would allow for better patient compliance.

### SUMMARY

The present disclosure is directed to compositions and methods for using the compositions in the treatment of pulmonary hypertension. In embodiments herewith, a composition is provided in a dry powder inhaler comprising a replaceable cartridge comprising a dry powder for inhalation for delivery to the lungs for local or systemic delivery into the pulmonary circulation. The dry powder inhaler is a breath-powered inhaler which is compact, reusable or disposable, has various shapes and sizes, and comprises a system of airflow conduit pathways for the effective and rapid delivery of powder medicament to the lungs and the systemic circulation.

In a particular embodiment, the method of treating pulmonary arterial hypertension utilizes a drug delivery system which is designed for drug delivery to the lungs, including by inhalation, for rapid delivery and onset of action of the active agent being delivered to target tissues using the arterial circulation in the lungs. In this method, the active agent can reach its target site in a therapeutically effective manner.

In one embodiment, the method comprises administering a stable pharmaceutical composition comprising, one or more active agents, including, a vasodilator, including, sildenafil, tadalafil, vardenafil, a prostaglandin or an analog thereof, for example, treprostinil or a pharmaceutically acceptable salt thereof, including treprostinil sodium, for treating PAH and delivering the treprostinil into the systemic circulation of a subject by pulmonary inhalation using a dry powder inhaler. In one embodiment, the method comprises providing to a patient in need of treatment a dry powder inhaler comprising treprostinil in a stable dry powder formulation, and administering the active agent by oral inhalation.

In one embodiment, the drug delivery system comprises a dry powder inhaler comprising a diketopiperazine-based drug formulation for delivering small molecules, for example, a prostaglandin, or analogs thereof including, tresprostinil and protein-based products for treating PAH. The method provides advantages over typical methods of drug delivery, such as, oral tablet and subcutaneous and intravenous injectable/infusion drug products that are sensitive to degradation and/or enzymatic deactivation.

In certain embodiments disclosed herein, a method for providing a prostaglandin formulation to a patient in need thereof is disclosed, the method comprising, selecting a patient to be treated for PAH patient, and administering to the patient a dry powder formulation comprising treprostinil; wherein the treprostinil is combined with a diketopiperazine to produce a pharmaceutical formulation or composition suitable for pulmonary inhalation, and delivering the trepostinil formulation using a breath-powered dry powder inhaler. In this and other embodiments, the dry powder formulations is provided in a reconfigurable cartridge comprising from about 1 μg to about 200 μg of treprostinil in the dry powder formulation per dose. In certain embodiments, the dry powder formulation can comprise from about 10 μg to about 300 μg of treprostinil per dose in a cartridge or capsule. In one embodiment, a cartridge for single use can comprise from about 10 μg to about 90 μg of treprostinil for at least one inhalation. In some embodiments, the dry powder formulation is delivered using at least one inhalation per use. In this and other embodiments, the dry powder formulation is delivered to a patient in less than 10 seconds, or less than 8 seconds or less than 6 seconds per inhalation or breath. In one embodiment, the pharmaceutical dry powder composition comprises microcrystalline particles of fumaryl diketopiperazine wherein the particles have a specific surface area ranging from about 59 m²/g to about 63 m²/g and have a pore size ranging from about 23 nm to about 30 nm.

Also disclosed herein is a method of treating a pulmonary arterial hypertension disease or disorder comprising, selecting a patient to be treated with pulmonary arterial hypertension, or a patient with PAH which exhibits a condition treatable with an active agent, including treprostinil, epoprostenol, bosentan, ambrisentan, macisentan, sildenafil, tadalafil, riociguat and the like, or combinations thereof, which patients are typically treated only by oral or injectable administration; replacing the aforementioned therapy with an inhalation therapy comprising providing the patient with an inhaler comprising the active agent in a stable dry powder composition for treating the disease or disorder; wherein the stable dry powder composition comprises the active agent and a diketopiperazine; and administering the stable dry powder composition to the patient by pulmonary inhalation; thereby treating the disease or condition.

In an exemplary embodiment, the formulation for treating pulmonary arterial hypertension comprises treprostinil in an amount up to 200 ∞g per dose, for example, amounts of 1 μg, 5 μg, 10 μg, 15 μg, 20 ∞g, 30 μg, 60 μg, 90 μg, 100 μg, 120 μg, 150 μg, 180 μg, or 200 μg, and one or more pharmaceutically acceptable carriers and/or excipients per dose are to be administered to a subject. In this embodiment, the pharmaceutically acceptable carrier and/or excipient can be formulated for oral inhalation and can form particles, for example, a diketopiperazine, including, fumaryl diketopiperazine, sugars such as mannitol, xylitol, sorbitol, and trehalose; amino acids, including, glycine, leucine, isoleucine, methionine; surfactants, including, polysorbate 80; cationic salts, including, monovalent, divalent and trivalent salts, including, sodium chloride, potassium chloride, magnesium chloride, and zinc chloride; buffers such as citrates and tartrates, or combination of one or more carriers and/or excipients and the like. In a particular embodiment, the formulation comprises a dry powder comprising treprostinil, a sugar and an amino acid, wherein the sugar is mannitol or trehalose; and the amino acid is leucine or isoleucine and a cationic salt. In certain embodiments, the formulation can further comprise sodium chloride, potassium chloride, magnesium chloride or zinc chloride, sodium citrate, sodium tartrate, or combinations thereof.

In an exemplary embodiment, the treprostinil dose is administered using a dry powder inhaler for oral inhalation. In this embodiment, a treprostinil inhalation powder dose is provided to a patient suffering with pulmonary arterial hypertension and in need of treatment; wherein the a dry powder inhaler comprises a container including, a cartridge, and the container or cartridge comprises the dry powder comprising treprostinil is administered in multiple daily doses for a period of six months and the treprostinil is administered by oral inhalation at an earlier time in the course of the disease to patients with Functional Class II as a first line monotherapy.

In one embodiment, a method for treating pulmonary arterial hypertension is provided comprising providing a patient in need of treatment a monotherapy using an inhalable dry powder comprising treprostinil and a pharmaceutically acceptable carrier, and/or excipient by oral inhalation using a dry powder inhaler and a container comprising the inhalable dry powder and administering the dry powder formulation to the patient. In some embodiments, the treprostinil formulation comprises fumaryl diketopiperazine particles.

In one embodiment, a method for treating pulmonary arterial hypertension is provided comprising providing a patient in need of treatment a combination therapy using an inhalable dry powder comprising treprostinil and fumaryl diketopiperazine, and administering separately in combination with orally administered drugs selected from prostacyclin analogues, endothelin receptor antagonists (ERAs), including bosentran, ambrisentran and macitentan, soluble guanine cyclase agonists/stimulators such as riociguat, and PDE-5 inhibitors, including sildenafil, vardenafil and tadalafil.

In another embodiment, a dry powder comprising treprostinil and fumaryl diketopiperazine can also be administered as a part of up-front combination therapy with an oral agent. In an alternate embodiment, an inhalable treprostinil composition comprising a dose of fumaryl diketopiperazine and treprostinil powder, wherein treprostinil is in an amount from about 1 μg to about 200 μg administered in combination with an oral agent such as a PDE-5 inhibitor, or an endothelin receptor antagonist and/or the combination therapy may also be administered to replace continuously parenteral infusion of prostacyclin analogs in patients with severe disease and classified in WHO Functional class IV. Phosphodiesterase inhibitors, including PDE-5 inhibitors can also be formulated for inhalation alone, or in combination with the treprostinil and can be administered subsequently if administered alone, as a combination therapy.

In another embodiment, the inhalation system comprises a breath-powered dry powder inhaler, a container or cartridge containing a dry powder, for delivering an active agent to the pulmonary tract and lungs, including a medicament, wherein the medicament can comprise, for example, an inhalable drug formulation for pulmonary delivery such as a composition comprising a diketopiperazine in a crystalline powder form that self-assembles in a suspension, an amorphous powder form, and/or a microcrystalline powder form comprising crystallites that do not self-assemble in suspension, or combinations thereof, and an active agent, including, treprostinil, sildenafil, vardenafil, tadalafil, or combinations thereof.

In alternate embodiments, the dry powder for inhalation may be formulated with other carriers and/or excipients other than diketopiperazines, for example a sugar, including trehalose; buffers, including sodium citrate; salts, including, sodium chloride and zinc chloride, and one or more active agents, including, treprostinil, vardenafil, and sildenafil.

In embodiments herewith, the method of treating PAH comprises, administering to a patient with moderate to severe PAH a dry powder formulation comprising treprostinil and a pharmaceutically acceptable carrier and/or excipient in an amount up to 200 μg of treprostinil using a dry powder inhaler comprising a movable member for loading a container comprising the pharmaceutical composition and the movable member can configure a container to attain a dosing configuration from a container loading configuration so that inhaler creates an airflow through the inhaler during an inhalation maneuver to allow the contents of the container to enter the airflow path and greater than 60% of a dry powder dose in the container is delivered to the lungs in a single inhalation.

In some embodiments, the treatment regimen with an inhalation dry powder depends on the patient's need and can be one inhalation to replace each of a nebulization session performed with standard therapy, including, at least one to four inhalations per day depending on the severity of disease.

### DETAILED DESCRIPTION

In embodiments disclosed herein, dry powder compositions and dry powder inhalers comprising a container or a cartridge for delivering dry powders including pharmaceutical medicaments to a subject by oral inhalation are described. In one embodiment, the dry powder inhaler is a breath-powered, dry powder inhaler, and the container or cartridge is designed to contain an inhalable dry powder, including but not limited to pharmaceutical formulations comprising an active ingredient, including a pharmaceutically active substance, and optionally, a pharmaceutically acceptable carrier. In particular, the dry powder inhalers are for the treatment of pulmonary arterial hypertension.

The dry powder inhalers are provided in various embodiments of shapes and sizes, and can be reusable, easy to use, inexpensive to manufacture and/or produced in high volumes in simple steps using plastics or other acceptable materials. Various embodiments of the dry powder inhalers are provided herein and in general, the inhalation systems comprise inhalers, powder-filled cartridges, and empty cartridges. The present inhalation systems can be designed to be used with any type of dry powder. In one embodiment, the dry powder is a relatively cohesive powder which requires optimal deagglomeration conditions. In one embodiment, the inhalation system provides a re-useable, miniature breath-powered inhaler in combination with single-use cartridges containing pre-metered doses of a dry powder formulation. The inhaler can deliver a dry powder dose in a single inhalation to a patient in treating pulmonary arterial hypertension in less than 10 seconds. In particular embodiments, oral inhalation can deliver greater than 60% of a powder dose in less than 6 seconds, in less than 4 seconds and in less than 2 seconds.

As used herein the term “a unit dose inhaler” refers to an inhaler that is adapted to receive a single enclosure, cartridge or container comprising a dry powder formulation and delivers a single dose of a dry powder formulation by inhalation from a single container to a user. It should be understood that in some instances multiple unit doses will be required to provide a user with a specified dosage.

As used herein a “cartridge” is an enclosure configured to hold or contain a dry powder formulation, a powder containing enclosure, which has a cup or container and a lid. The cartridge is made of rigid materials, and the cup or container is moveable relative to the lid in a translational motion or vice versa.

As used herein a “powder mass” is referred to an agglomeration of powder particles or agglomerate having irregular geometries such as width, diameter, and length.

As used herein a “unit dose” refers to a pre-metered dry powder formulation for inhalation. Alternatively, a unit dose can be a single enclosure including a container having a single dose or multiple doses of formulation that can be delivered by inhalation as metered single amounts. A unit dose enclosure/cartridge/container contains a single dose. Alternatively it can comprise multiple individually accessible compartments, each containing a unit dose.

As used herein, the term “about” is used to indicate that a value includes the standard deviation of error for the device or method being employed to determine the value.

As used herein, the term “microparticle” refers to a particle with a diameter of about 0.5 to about 1000 irrespective of the precise exterior or interior structure. Microparticles having a diameter of between about 0.5 and about 10 microns can reach the lungs, successfully passing most of the natural barriers. A diameter of less than about 10 microns is required to navigate the turn of the throat and a diameter of about 0.5 μm or greater is required to avoid being exhaled. To reach the deep lung (or alveolar region) where most efficient absorption is believed to occur, it is preferred to maximize the proportion of particles contained in the “respirable fraction” (RF), generally accepted to be those particles with an aerodynamic diameter of about 0.5 to about 6 though some references use somewhat different ranges, as measured using standard techniques, for example, with an Anderson Cascade Impactor. Other impactors can be used to measure aerodynamic particle size such as the NEXT GENERATION IMPACTOR™ (NGI™, MSP Corporation), for which the respirable fraction is defined by similar aerodynamic size, for example <6.4 μm. In some embodiments, a laser diffraction apparatus is used to determine particle size, for example, the laser diffraction apparatus disclosed in U.S. Pat. No. 8,508732, which disclosure is incorporated herein in its entirety for its relevant teachings related to laser diffraction, wherein the volumetric median geometric diameter (VMGD) of the particles is measured to assess performance of the inhalation system. For example, in various embodiments cartridge emptying of ≥80%, 85%, or 90% and a VMGD of the emitted particles of <12.5 μm, <7.0 μm, or <4.8 μm can indicate progressively better aerodynamic performance.

Respirable fraction on fill (RF/fill) represents the percentage (%) of powder in a dose that is emitted from an inhaler upon discharge of the powder content filled for use as the dose, and that is suitable for respiration, i.e., the percent of particles from the filled dose that are emitted with sizes suitable for pulmonary delivery, which is a measure of microparticle aerodynamic performance. As described herein, a RF/fill value of 40% or greater than 40% reflects acceptable aerodynamic performance characteristics. In certain embodiments disclosed herein, the respirable fraction on fill can be greater than 50%. In an exemplary embodiment, a respirable fraction on fill can be up to about 80%, wherein about 80% of the fill is emitted with particle sizes <5.8 μm as measured using standard techniques.

As used herein, the term “dry powder” refers to a fine particulate composition that is not suspended or dissolved in a propellant, or other liquid. It is not meant to necessarily imply a complete absence of all water molecules.

As used herein, “amorphous powder” refers to dry powders lacking a definite repeating form, shape, or structure, including all non-crystalline powders.

The present disclosure also provides improved powders comprising microcrystalline particles, compositions, methods of making the particles, and therapeutic methods that allow for improved delivery of drugs to the lungs for treating diseases and disorders in a subject. Embodiments disclosed herein achieve improved delivery by providing crystalline diketopiperazine compositions comprising microcrystalline diketopiperazine particles having high capacity for drug adsorption yielding powders having high drug content of one or more active agents. Powders made with the present microcrystalline particles can deliver increased drug content in lesser amounts of powder dose, which can facilitate drug delivery to a patient. The powders can be made by various methods including, methods utilizing surfactant-free solutions or solutions comprising surfactants depending on the starting materials.

In alternate embodiments disclosed herein, the drug delivery system can comprise a dry powder for inhalation comprising a plurality of substantially uniform, microcrystalline particles, wherein the microcrystalline particles can have a substantially hollow spherical structure and comprise a shell which can be porous comprising crystallites of a diketopiperazine that do not self-assemble in a suspension or in solution. In certain embodiments, the microcrystalline particles can be substantially hollow spherical and substantially solid particles comprising crystallites of the diketopiperazine depending on the drug and/or drug content provided and other factors in the process of making the powders. In one embodiment, the microcrystalline particles comprise particles that are relatively porous, having average pore volumes of about 0.43 cm³/g, ranging from about 0.4 cm³/g to about 0.45 cm³/g, and average pore size ranging from about 23 nm to about 30 nm, or from about 23.8 nm to 26.2 nm as determined by BJH adsorption.

Certain embodiments disclosed herein comprise dry powders comprising a plurality of substantially uniform, microcrystalline particles, wherein the particles have a substantially spherical structure comprising a shell which can be porous, and the particles comprise crystallites of a diketopiperazine that do not self-assemble in suspension or solution, and have a volumetric median geometric diameter less than 5 μm; or less than 2.5 μm and comprise an active agent.

In a particular embodiment herein, up to about 92% of the microcrystalline particles have a volumetric median geometric diameter of 5.8 μm. In one embodiment, the particle's shell is constructed from interlocking diketopiperazine microcrystals having one or more drugs adsorbed on their surfaces. In some embodiments, the particles can entrap the drug in their interior void volume and/or combinations of the drug adsorbed to the crystallites' surface and drug entrapped in the interior void volume of the spheres.

In certain embodiments, a diketopiperazine composition comprising a plurality of substantially uniformly formed, microcrystalline particles is provided, wherein the particles have a substantially hollow spherical structure and comprise a shell comprising crystallites of a diketopiperazine that do not self-assemble; wherein the particles are formed by a method comprising the step of combining diketopiperazine having a trans isomer content ranging from about 45% to 65% in a solution and a solution of acetic acid without the presence of a surfactant and concurrently homogenizing in a high shear mixer at high pressures of up to 2,000 psi to form a precipitate; washing the precipitate in suspension with deionized water; concentrating the suspension and drying the suspension in a spray drying apparatus. The microcrystalline particles can be pre-formed without for later used, or combined with an active agent in suspension prior to spray drying.

The method can further comprise the steps of adding with mixing a solution comprising an active agent or an active ingredient such as a drug or bioactive agent along with other pharmaceutically acceptable carriers and/or excipients prior to the spray drying step so that the active agent or active ingredient is adsorbed and/or entrapped on or within the particles. Particles made by this process can be in the submicron size range prior to spray-drying.

In certain embodiments, a diketopiperazine composition comprising a plurality of substantially uniformly formed, microcrystalline particles is provided, wherein the particles have a substantially hollow spherical structure and comprise a shell comprising crystallites of a diketopiperazine that do not self-assemble, and the particles have a volumetric mean geometric diameter less than equal to 5 μm; wherein the particles are formed by a method comprising the step of combining diketopiperazine in a solution and a solution of acetic acid without the presence of a surfactant and concurrently homogenizing in a high shear mixer at high pressures of up to 2,000 psi to form a precipitate; washing the precipitate in suspension with deionized water; concentrating the suspension and drying the suspension in a spray drying apparatus.

The method can further comprise the steps of adding with mixing a solution comprising an active agent or an active ingredient such as a drug or bioactive agent prior to the spray drying step so that the active agent or active ingredient is adsorbed and/or entrapped on or within the particles. Particles made by this process can be in the submicron size range prior to spray-drying.

In certain embodiments, a diketopiperazine composition comprising a plurality of substantially uniformly formed, microcrystalline particles is provided, wherein the microcrystalline particles have a substantially hollow spherical structure and comprise a shell comprising crystallites of a diketopiperazine that do not self-assemble, and the particles have a volumetric mean geometric diameter less than equal to 5 μm; wherein the particles are formed by a method comprising the step of combining diketopiperazine in a solution and a solution of acetic acid without the presence of a surfactant and without the presence of an active agent, and concurrently homogenizing in a high shear mixer at high pressures of up to 2,000 psi to form a precipitate; washing the precipitate in suspension with deionized water; concentrating the suspension and drying the suspension in a spray drying apparatus.

In certain embodiments wherein the starting material comprising the active ingredient is an extract exhibiting a high degree of viscocity, or a substance having a honey like viscous appearance, the microcrystalline particles are formed as above and by washing them in water using tangential flow filtration prior to combining with the extract or viscous material. After washing in water, the resultant particle suspension is lyophilized to remove the water and re-suspended in an alcohol solution, including ethanol or methanol prior to adding the active ingredient as a solid, or in a suspension, or in solution. In one embodiment, optionally, the method of making the composition comprises the step of adding any additional excipient, including one or more, amino acid, such as leucine, isoleucine, norleucine, methionine or one or more phospholipids, for example, 1,2-dipalmitoyl-sn-glycero-3-phosphocholine (DPPC) or 1,2-distearoyl-sn-glycero-3-phosphocholine (DSPC), concurrently with the active ingredient or subsequent to adding the active ingredient, and prior to spray drying. In certain embodiments, forming the composition comprises the step wherein the extract comprising desired active agents is optionally filtered or winterized to separate and remove layers of unwanted materials such as lipids to increase its solubility.

The method can further comprise the steps of adding a solution with mixing to the mixture, and wherein the mixing can optionally be performed with or without homogenization in a high shear mixer, wherein the solution comprises an active agent or an active ingredient such as a drug or bioactive agent prior to the spray drying step so that the active agent or active ingredient is adsorbed and/or entrapped within or on the surface of the particles. Particles made by this process can be in the submicron size range prior to spray-drying, or the particles can be formed from the solution during spray-drying.

In some embodiments herewith, the drug content can be delivered on crystalline powders using FDKP and which are lyophilized or sprayed dried at contents to about 10%, or about 20%, or about 30% or higher. In embodiments using microcrystalline particles formed from FDKP, or FDKP disodium salt, and wherein the particles do not self-assemble and comprise submicron size particles, drug content can typically be greater than 0.01% (w/w). In one embodiment, the drug content to be delivered with the microcrystalline particles of from about 0.01% (w/w) to about 75 (w/w); from about 1% to about 50% (w/w), from about 10% (w/w) to about 25% (w/w), or from about 10% to about 20% (w/w), or from 5% to about 30%, or greater than 25% depending on the drug to be delivered. An example embodiment wherein the drug is a peptide such as insulin, the present microparticles typically comprise approximately 10% to 45% (w/w), or from about 10% to about 20% (w/w) insulin. In certain embodiments, the drug content of the particles can vary depending on the form and size of the drug to be delivered.

In an exemplary embodiment, the composition comprises a dry powder comprising microcrystalline particles of fumaryl diketopiperazine, wherein the treprostinil is adsorbed to the particles and wherein the content of the treprostinil in the composition comprises up to about 20% (w/w) and ranges from about 0.5% to about 10% (w/w), or from about 1% to about 5% (w/w) of the dry powder. In one embodiment, the composition herein can comprise other excipients suitable for inhalation such as amino acids including methionine, isoleucine and leucine. In this embodiment, the treprostinil composition can be used in the prevention and treatment of pulmonary hypertension by self-administering an effective dose comprising about 1 mg to 15 mg of a dry powder composition comprising microcrystalline particles of fumaryl diketopiperazine and treprostinil in a single inhalation. In a particular embodiment, the treprostinil content in the formulation can be from about 1 μg to about 200 μg. In one embodiment, the dry powder content of the cartridges comprising treprostinil can be 20 μg, 30 μg, 60 μg, 90 μ_{g,} 120 μg, 150 μg, 180 μg, or 200 μg.

In alternate embodiments, the pharmaceutically acceptable carrier for making dry powders can comprise any carriers or excipients useful for making dry powders and which are suitable for pulmonary delivery. Example of pharmaceutically suitable carriers and excipients include, sugars, including saccharides and polysaccharides, such as lactose, mannose, sucrose, mannitol, trehalose; citrates, amino acids such as glycine, L-leucine, isoleucine, trileucine, tartrates, methionine, vitamin A, vitamin E, zinc citrate, sodium citrate, trisodium citrate, sodium tartrate, sodium chloride, zinc chloride, zinc tartrate, polyvinylpyrrolidone, polysorbate 80, phospholipids including diphosphotidylcholine and the like.

In one embodiment, a method of self-administering a dry powder formulation to one's lung(s) with a dry powder inhalation system is also provided. The method comprises: obtaining a dry powder inhaler in a closed position and having a mouthpiece; obtaining a cartridge comprising a pre-metered dose of a dry powder formulation in a containment configuration; opening the dry powder inhaler to install the cartridge; closing the inhaler to effectuate movement of the cartridge to a dose position; placing the mouthpiece in one's mouth, and inhaling once deeply to deliver the dry powder formulation.

In still yet a further embodiment, a method of treating obesity, hyperglycemia, insulin resistance, pulmonary hypertention, anaphylaxis, and/or diabetes is disclosed. The method comprises the administration of an inhalable dry powder composition or formulation comprising, for example, a diketopiperazine having the formula 2,5-diketo-3,6-di(4-X-aminobutyl)piperazine, wherein X is selected from the group consisting of succinyl, glutaryl, maleyl, and fumaryl. In this embodiment, the dry powder composition can comprise a diketopiperazine salt. In still yet another embodiment, there is provided a dry powder composition or formulation, wherein the diketopiperazine is 2,5-diketo-3,6-di-(4-fumaryl-aminobutyl)piperazine, with or without a pharmaceutically acceptable carrier, or excipient.

An inhalation system for delivering a dry powder formulation to a patient's lung(s) is provided, the system comprising a dry powder inhaler configured to have flow conduits with a total resistance to flow in a dosing configuration ranging in value from 0.065 to about 0.200 (√kPa)/liter per minute. The dry powder inhaler can be provided comprising a dry powder formulation for single use that can be discarded after use, or with individual doses that are replaceable in a multiple use inhaler and the individual dose enclosures or containers can be discarded after use.

In one embodiment, a dry powder inhalation kit is provided comprising a dry powder inhaler as described above, one or more medicament cartridges comprising a dry powder formulation for treating a disorder or disease such as respiratory tract and lung disease, including pulmonary arterial hypertension, cystic fibrosis, respiratory infections, cancer, and other systemic diseases, including, endocrine disease, including, diabetes and obesity.

Methods of treating a disease or disorder in a patient with the dry powder inhaler embodiments disclosed herewith is also provided. The method of treatment comprises providing to a patient in need of treatment a dry powder inhaler comprising a cartridge containing a dose of an inhalable formulation comprising an active ingredient selected from the group as described above and a pharmaceutical acceptable carrier and/or excipient; and having the patient inhale through the dry powder inhaler deeply for about 3 to 4 seconds to deliver the dose. In the method, the patient can resume normal breathing pattern thereafter.

The following examples illustrate some of the processes for making dry powders suitable for using with the inhalers described herein and data obtained from experiments using the dry powders.

### EXAMPLE 1

Preparation of surfactant-free dry powder comprising FDKP microcrystalline powder for use with inhalers: In an example embodiment, surfactant free dry-powders comprising FDKP microcrystalline particles were prepared. Using a dual-feed high shear mixer, approximately equal masses of acetic acid solution (Table 1) and FDKP solution (Table 2) held at about 25° C.±5° C. were fed at 2000 psi through a 0.001-in² orifice to form a precipitate by homogenization. The precipitate was collected in deionized (DI) water of about equal temperature. The wt % content of FDKP microcrystallites in the suspension is about 2-3.5%. The suspension FDKP concentration can be assayed for solids content by an oven drying method. The FDKP microcrystallite suspension can be optionally washed by tangential flow filtration using deionized water. The FDKP microcrystallites can be optionally isolated by filtration, centrifugation, spray drying or lyophilization.

TABLE 1
Composition of Acetic Acid Solution

| Component | Component Range (wt. %) |
|---|---|
| Acetic Acid | 10.5-13.0 |
| Deionized Water | 87.0-89.5 |

TABLE 2
Composition of FDKP Solution

| Component | Component Range (wt. %) |
|---|---|
| FDKP | 2.5-6.25 |
| 30% NH4OH Solution | 1.6-1.75 |
| Deionized Water | 92-95.9 |

Dry powders (A, B, C and D) comprising microcrystalline particles made by the methods described above were tested for various characteristics, including surface area, water content and porosity measurements. Four different powders were used in this experiments. All powders tested had a residual water content of 0.4%. Table 2a demonstrates data obtained from the experiments.

TABLE 2a

| | | Pore Volume | Pore Size |
|---|---|---|---|
| | Surface Area | BJH Adsorption | BJH Adsorption |
| | BET Surface | cumulative volume of | average pore |
| Powder ID | Area (m²/g) | pores (cm³/g) | diameter (4 V/A) (nm) |
| A | 61.3 | 0.43 | 25.1 |
| B | 62.3 | 0.43 | 24.4 |
| C | 63.0 | 0.42 | 23.8 |
| D | 59.0 | 0.44 | 26.2 |

The data in Table 2a show that the surface area of sprayed-dried, bulk dry powder comprising the microcrystalline particles of the samples tested ranged from 59 m²/g to 63 m²/g. The porosity data indicate that the microcrystalline particles are relatively porous, having average pore volumes of about 0.43 cm³/g and average pore size ranging from about 23.8 nm to 26.2 nm as determined by BJH adsorption. The porosimetry data indicate that these particles differ from prior art FDKP microparticles which have been shown to have an average pore volume of about 0.36 cm³/g and average pore size from about 20 nm to about 22.6 nm.

### EXAMPLE 2

Preparation of dry powder comprising microcrystalline FDKP particles containing treprostinil. A solution containing 0.2-1.0 wt% treprostinil in ethyl alcohol was added to a suspension of FDKP microcrystallites obtained as described in Example 1. The mixture was spray dried using a Buchi B290 spray-dryer equipped with a high efficiency cyclone. Nitrogen was used as the process gas (60 mm). Mixture were dried using 10-12% pump capacity, 90-100% aspiration rate, and an inlet temperature of 170-190° C. The weight % concentration of treprostinil in the resultant powder was 0.5-10%. Delivery efficiencies of these powders after discharge from a dry powder inhaler ranged between approximately 50% and 70%.

### EXAMPLE 3

Use of treprostinil-fumaryl diketopiperazine composition in healthy subjects. This study was an open-label, single ascending dose study in 36 healthy normal volunteers that were sequentially assigned to 6 cohorts receiving single doses of TreT (30, 60, 90, 120, 150, and 180 μg). The safety and tolerability of the dry powder compositions comprising treprostinil was evaluated in each sequential cohort prior to escalating the dose for the next cohort using a dry powder inhaler system comprising a cartridge dose in a single inhalation. Blood samples were obtained before administration of the composition and at selected times through 480 minutes post-dose. Blood samples were analyzed for treprostinil using a validated analytical method and PK parameters were calculated using non-compartmental methods.

A total of 36 individuals were randomized and dosed. There were no severe adverse events, serious adverse events, or deaths during this study. No adverse events led to a subject's early termination. The most frequently reported adverse events were cough (n=11, 30.6%) and headache (n=8, 22%). Bioanalysis data confirmed that the treprostinil plasma concentrations and exposure for treprostinil, achieved clinically relevant concentrations comparable to those observed in historical Tyvaso® single dose clinical studies. Cmax and AUC for treprostinil, increased in a linear manner with increasing dose. Overall, treprostinil was safe and well-tolerated and produced clinically relevant concentrations of treprostinil when inhaled as a dry powder.

The preceding disclosures are illustrative embodiments. It should be appreciated by those of skill in the art that the devices, techniques and methods disclosed herein elucidate representative embodiments that function well in the practice of the present disclosure. However, those of skill in the art should, in light of the present disclosure, appreciate that many changes can be made in the specific embodiments that are disclosed and still obtain a like or similar result without departing from the spirit and scope of the invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope otherwise claimed. No language in the specification should be construed as indicating any non-claimed element essential to the practice of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments are described herein, including the best mode known to the inventors for carrying out the invention. Of course, variations on those preferred embodiments will become apparent to those of ordinary skill in the art upon reading the foregoing description. The inventor expects those of ordinary skill in the art to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments so claimed are inherently or expressly described and enabled herein.

Furthermore, numerous references have been made to patents and printed publications throughout this specification. Each of the above cited references and printed publications are herein individually incorporated by reference in their entirety.

Further, it is to be understood that the embodiments disclosed herein are illustrative of the principles of the present invention. Other modifications that may be employed are within the scope of the invention. Thus, by way of example, but not of limitation, alternative configurations may be utilized in accordance with the teachings herein. Accordingly, the present invention is not limited to that precisely as shown and described.

We claim:

1. A pharmaceutical dry powder composition comprising a treprostinil dose in an amount of up to 200 μg and one or more pharmaceutically acceptable salt thereof and a pharmaceutically acceptable carrier and/or excipients.

2. The pharmaceutical dry powder composition of claim 1, wherein the one or more pharmaceutically acceptable carrier and/or excipient is a diketopiperazine.

3. The pharmaceutical dry powder composition of claim 2, wherein the diketopiperazine is fumaryl diketopiperazine and comprises microcrystalline particles comprising crystallites of the diketopiperazine and the tresprostinil.

4. The pharmaceutical dry powder composition of claim 1, wherein the treprostinil is from about 1 μg to about 180 μg in the dry powder composition.

5. The pharmaceutical dry powder composition of claim 1, wherein the pharmaceutical dry powder composition is in substantially crystalline form.

6. The pharmaceutical dry powder composition of claim 1, wherein the one or more pharmaceutically acceptable carrier and/or excipients is selected from lactose, mannose, sucrose, mannitol, trehalose, sodium citrate, trisodium citrate, zinc citrate, glycine, L-leucine, isoleucine, trileucine, sodium tartrate, zinc tartrate, methionine, vitamin A, vitamin E, sodium chloride, zinc chloride, polyvinylpyrrolidone, or polysorbate 80.

7. The pharmaceutical dry powder composition of claim 6, wherein the one or more pharmaceutically acceptable carrier and/or excipient are sodium citrate, sodium chloride, leucine or isoleucine, and trehalose.

8. The pharmaceutical dry powder composition of claim 7, further comprising polysorbate 80.

9. The pharmaceutical dry powder composition of claim 3, wherein microcrystalline particles have a specific surface area ranging from about 59 m²/g to about 63 m²/g.

10. The pharmaceutical dry powder composition of claim 3, wherein microcrystalline particles have a pore size ranging from about 23 nm to about 30 nm.

11. A method of treating pulmonary arterial hypertension comprising administering to a patient in need of treatment by oral inhalation using a dry powder inhaler comprising a dry powder composition comprising up to 200 μg of treprostinil or a pharmaceutically acceptable salt thereof, and/or one or more pharmaceutically acceptable carrier and/or excipient.

12. The method of treating pulmonary arterial hypertension of claim 11, wherein the one or more pharmaceutically acceptable carrier and/or excipients is selected from the group consisting of fumaryl diketopiperazine, lactose, mannose, sucrose, mannitol, trehalose, sodium citrate, trisodium citrate, zinc citrate, glycine, L-leucine, isoleucine, trileucine, sodium tartrate, zinc tartrate, methionine, vitamin A, vitamin E, sodium chloride, zinc chloride, polyvinylpyrrolidone, and polysorbate 80.

13. The method of treating pulmonary arterial hypertension of claim 12, wherein the one or more pharmaceutically acceptable carrier and/or excipient are sodium citrate, sodium chloride, leucine or isoleucine, and trehalose.

14. The method of treating pulmonary arterial hypertension of claim 11, wherein the one or more pharmaceutically acceptable carrier and/or excipient is fumaryl dikepiperazine.

15. The method of treating pulmonary arterial hypertension of claim 11, wherein the dry powder composition is administered in at least one inhalation in less than 10 seconds.

16. A dry powder inhaler comprising a movable member to load an enclosure and configure the container to attain a dosing configuration, wherein said enclosure comprises the pharmaceutical dry powder composition of claim 1.

17. The dry powder inhaler of claim 16, wherein the enclosure comprises a cartridge consisting of a lid and a container.
